(19) United States
(12) Patent Application Publication
(10) Pub. No.: US 2009/0156887 A1
(43) Pub. Date: Jun. 18, 2009

(54) SYSTEM AND METHOD FOR PERCEIVING AND RELAXING EMOTIONS

(75) Inventor: Chih-Wei HSU, Tainan County (TW)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: INSTITUTE FOR INFORMATION INDUSTRY, Taipei City (TW)

(21) Appl. No.: 12/052,347

(22) Filed: Mar. 20, 2008

(30) Foreign Application Priority Data
Dec. 12, 2007 (TW) 096147547

(51) Int. Cl. A61M 21/00

(52) U.S. Cl. 600/27

(57) ABSTRACT

A system and method for perceiving and relaxing emotions are described. The system includes a biosensor, an emotion database, a media player, and an emotion analysis module. According to the method, first, the biosensor detects bio-signals from an individual. Next, the emotion analysis module analyzes the bio-signals to construct a 3D emotional coordinate system, then calculates the position of the bio-signals in the 3D emotional coordinate system to determine the emotional state of the individual, and obtains a media information corresponding to the emotional state information from the emotion database. Afterward, the media information is played by the media player to stabilize the emotion of the individual. In this manner, the emotion of the individual is controlled in a stable and safe way, thus preventing causing any physiological or inveterate diseases of the individual. This method is applicable to perform long-term medical monitoring and treatment on patients with chronic/acute diseases.

This application claims the benefit of Taiwan Patent Application No.096147547, filed on Jan. 9, 2008, which is hereby incorporated by reference for all purposes as if fully set forth herein.

### BACKGROUND OF THE INVENTION

1. Field of Invention

The present invention relates to a system for perceiving emotions, and more particularly to a system for perceiving and relaxing emotions that senses the emotion of an individual and plays a relative media information to release the emotion of the individual.

2. Related Art

In clinical experiments, the surface temperature of a finger is generally taken as an index for predicting the emotion or reaction of a human body, so as to facilitate the control of physiological or psychological diseases of the human body in relative medical treatments.

For example, Taiwan Utility Model Patent No. M312974, which is hereby incorporated by reference herein in its entirety, discloses a finger kit for sensing emotions which is applicable to a remote-control medical apparatus. The finger kit is further provided with a temperature sensing element for sensing the temperature of a finger, a processing element, and a transceiver connected with the remote end. Besides, the finger kit appears in a finger ring and is fitted on a finger to sense the temperature of the human body. Then, the temperature of the finger is outputted and displayed on a remote apparatus, such as a hand-held phone, personal digital assistant (PDA), hand-held computer, or personal computer (PC), for monitoring physiological conditions of the human body and determining emotional variations thereof in a long term. Thereby, medical personnel can adjust medical treatments based on the information of the received physiological conditions.

However, the prior art has the following unavoidable drawbacks.

First, for emotions of a human body, different emotions may result in different bio-signals, and the same temperature of the finger may be corresponding to more than two kinds of emotional expressions. For example, when the human body feels depressed or tense, the temperature of the finger is low, so if the emotion of the human body is determined merely according to the temperature of the finger, the result is surely inaccurate.

Secondly, as the temperature of the finger is detected to monitor the emotional variations of the human body, the medical personnel collect the required information after a long-term observation, and conclude and analyze the collected information to seek the best medical treatment for the human body. However, when the emotional variation of the human body is too abrupt, a means for releasing the emotion cannot be applied immediately.

### SUMMARY OF THE INVENTION

Accordingly, to solve the above problems, the present invention is directed to a system and method for perceiving and relaxing emotions, in which a plurality of types of bio-signals from an individual is collected and analyzed to obtain emotional states corresponding to the bio-signals, and a media information corresponding to the emotional state is played to release the emotion of the human body.

To solve the above problems in releasing the emotion of a human body, a system for perceiving and relaxing emotions is provided together with a method therefor, so as to release the emotion of an individual. The system includes a biosensor, an emotion database, a media player, and an emotion analysis module. The biosensor senses a plurality of types of bio-signals from an individual, and the emotion database stores a plurality of emotional state information and a plurality of media information.

According to the method, first, the biosensor detects a plurality of types of bio-signals from an individual, and the emotion analysis module identifies which emotional state information is corresponding to the bio-signals through analysis, so as to obtain a corresponding media information from the emotion database, and play the media information on the media player for releasing the emotion of the individual.

According to the system for perceiving and relaxing emotions provided by the present invention, the emotion analysis module constructs a 3D emotional coordinate system according to the bio-signals, and each of the emotional phases is corresponding to an emotional state information. Next, the emotion analysis module calculates the position of the bio-signals in the 3D emotional coordinate system, so as to obtain a corresponding emotional phase, and to analyze the emotional state of the individual accordingly. In addition, the emotional phase corresponding to each emotional state information is stored in the emotion database.

According to the system for perceiving and relaxing emotions provided by the present invention, after outputting the media information, the emotion analysis module continues to analyze variations of the bio-signals, and calculates an emotional quotient. As such, the media information corresponding to the emotional state information in the emotion database is modified when the emotional quotient shows that the individual is not in a good mood.

According to the system for perceiving and relaxing emotions provided by the present invention, the signals detected by the biosensor include temperature, heart rate, and SpO2 (Oxyhemoglobin saturation by pulse oximetry) of the individual.

Further, the method provided by the present invention is performed through a recording medium, in which computer programs recorded in the recording medium are read and executed to solve the same problems with the same effect.

The present invention is superior to the prior art in the following aspects.

First, a 3D emotional coordinate system is constructed based on a plurality of types of bio-signals, and each of the emotional phases is corresponding to a specific emotional reaction of the individual, such that the emotional state information of the individual can be reflected accurately and explicitly.

Secondly, the media information corresponding to the emotional state information is played to stabilize the emotion of the individual, so as to achieve a long-term and effective control of the emotional variations of the individual, and meanwhile reduce direct or indirect impacts on the physiology of the individual due to unstable emotions.

Thirdly, an emotional quotient is employed to determine whether the played media information can truly release the emotion of the individual. When the media information fails to release the emotion of the individual, the media information corresponding to the emotional state information is updated and variations of the bio-signals are again analyzed continuously, such that each emotional state information is corresponding to a media information for achieving the most stabilized emotion, thereby forming a self-training, progressing, and modifying model.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below for illustration only, and thus are not limitative of the present invention, and wherein:

FIG. 1 is a schematic view of an architecture of a system according to an embodiment of the present invention;

FIG. 2 is a flow chart of processes of perceiving and relaxing emotions according to an embodiment of the present invention;

FIG. 3 is a schematic view of a 3D emotional coordinate system according to an embodiment of the present invention;

FIG. 4 is a schematic view of a first configuration of the system according to an embodiment of the present invention; and

FIG. 5 is a schematic view of a second configuration of the system according to an embodiment of the present invention.

### DETAILED DESCRIPTION OF THE INVENTION

The objectives, structures, features, and functions of the present invention will be illustrated in detail below in embodiments with the accompanying drawings.

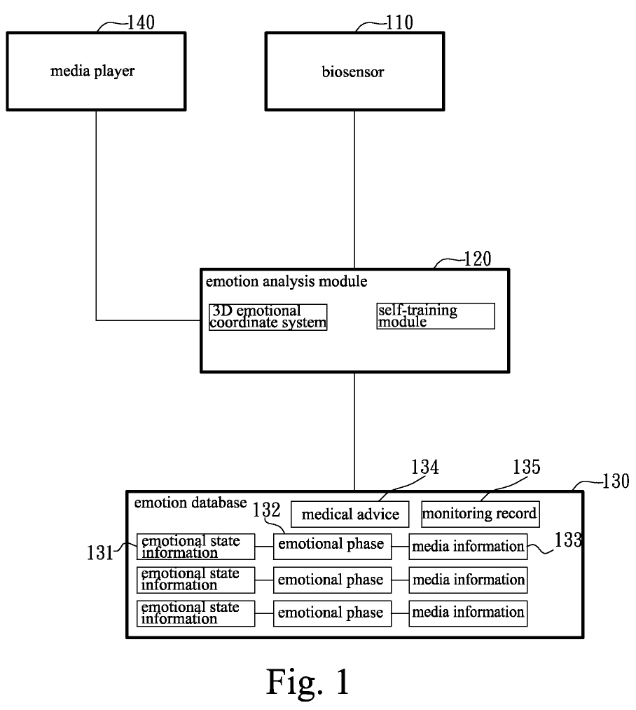

Referring to FIG. 1, a schematic view of an architecture of a system according to an embodiment of the present invention is shown. The system includes a biosensor 110, an emotion database 130, a media player 140, and an emotion analysis module 120. The emotion analysis module 120 is connected with the biosensor 110, the emotion database 130, and the media player 140. The biosensor 110 senses a plurality of types of bio-signals from an individual, and three types of bio-signals such as finger temperature, heart rate, and SpO2 (Oxyhemoglobin saturation by pulse oximetry) of an individual are taken as an example for illustration below. The emotion database 130 stores a plurality of emotional state information 131, a plurality of media information 133, and a plurality of emotional phases 132. Each emotional state information 131 is corresponding to one of the emotional phases 132 and one of the media information 133, and each of the emotional phases 132 further includes a particular range of values of the bio-signals. The emotion analysis module 120 analyzes the three types of bio-signals such as finger temperature, heart rate, and SpO2 of the individual, and constructs a 3D emotional coordinate system according to the three types of bio-signals. The 3D emotional coordinate system has various emotional phases 132, each representing an emotional reaction of the individual, such as anger, sadness, tension, calmness, excitement, happiness, depression, fear, and so on, i.e., reflecting the corresponding relationship between the emotional state information 131 and the emotional phases 132 stored in the emotion database 130. Then, the position of the bio-signals in the 3D emotional coordinate system is calculated to obtain the emotional phase 132 corresponding to the position. Therefore, the emotional state information 131 represented by the emotional phase 132 is acquired through comparison from the emotion database 130, and the corresponding media information 133 is then output. Thereafter, the media information 133 is played by the media player 140 to the individual, so as to stabilize the emotion of the individual.

The emotion analysis module 120 further includes a self-training module, for calculating the variation range of the bio-signals after the emotion analysis module 120 outputs the media information 133, so as to obtain an emotional quotient. In addition, the emotional quotient may be a positive or negative emotional quotient. The positive emotional quotient means that the media information 133 output by the emotion analysis module 120 is able to release the current emotion of the individual to a normal state. On the contrary, the negative emotional quotient means that the media information 133 output by the emotion analysis module 120 is unable to release the current emotion of the individual. Therefore, when the self-training module obtains a negative emotional quotient, the most appropriate media information 133 is acquired from all the media information 133 to substitute the media information 133 corresponding to the current emotional state information 131, and is stored in a monitoring record 135, so as to eliminate the media information 133, which is unable to release the emotion of the individual based on the determination of the self-training module, to be acquired again.

The emotion database 130 further stores at least one medical advice 134, for recording emotional reactions and variations that the individual may show or should avoid, and establishing optimal parameters for the bio-signals from the individual in the 3D emotional coordinate system, such that after obtaining the emotional state information 131 corresponding to the bio-signals, the emotion analysis module 120 calculates the media information 133 perfectly matching the emotional state information 131 with reference to the optimal parameters of the medical advice 134.

When referring to the medical advice 134, the emotion analysis module 120 analyzes the emotional state information 131 through a stochastic approximation method, so as to obtain the optimal parameters of the medical advice 134 that are closest and most consistent with the emotional state information 131, thereby acquiring the most appropriate media information 133 to be played. In addition, the media information 133 may be music or images.

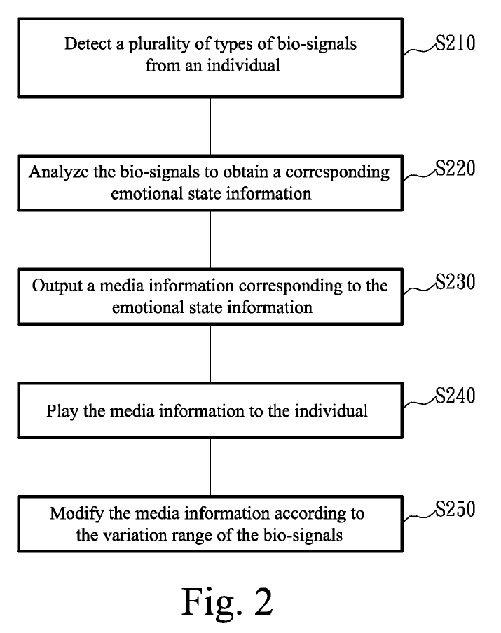

Referring to FIG. 2, a flow chart of processes of perceiving and relaxing emotions according to an embodiment of the present invention is shown. Based on the schematic view of the architecture of the system in FIG. 1, the method includes the following steps.

First, a plurality of bio-signals from an individual is detected (Step S210), in which the biosensor 110 detects a plurality of types of bio-signals from an individual, such as the aforementioned finger temperature, heart rate, and SpO2.

Next, the bio-signals are analyzed to obtain a corresponding emotional state information 131 (Step S220). After obtaining the bio-signals, the emotion analysis module 120 constructs a 3D emotional coordinate system with the bio-signals as coordinate axes, and divides the 3D emotional coordinate system into a plurality of emotional phases 132 according to the coordinate axes. Then, the emotion analysis module 120 sets each of the emotional phases 132 corresponding to an emotional state information 131, and calculates the emotional phase 132 of the bio-signals and the distance from the emotional phase to the origin of the 3D emotional coordinate system, so as to determine the emotional state information 131 corresponding to the bio-signals.

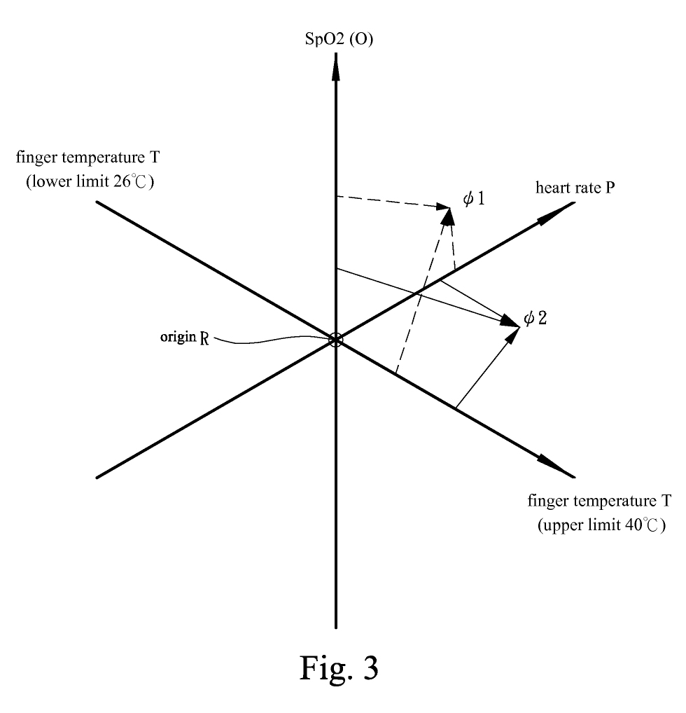

Referring to FIG. 3, the biosensor 110 constructs a 3D emotional coordinate system based on finger temperature (T), heart rate (P), and SpO2 (O), in which a vector coordinate is defined as (T, P, O). Moreover, the 3D emotional coordinate system should be constructed according to the bio-signals of a human body in normal condition. For example, an adult generally has a finger temperature of 36.5° C. to 37.0° C., a normal heart rate of 75 bpm, and a SpO2 lower than 95% of the blood, so a vector coordinate (37, 75, 95) is obtained. Therefore, the origin R takes (37, 75, 95) as its actual vector coordinate (0, 0, 0), and the actual vector coordinates of all the bio-signals are modified accordingly based on the vector coordinate (37, 75, 95) of the origin R, so as to establish a 3D emotional coordinate system suitable for the bio-signals.

However, normally, an individual has a finger temperature T of 26° C. to 40° C., a heart rate of 75 bpm to 150 bpm, and a SpO2 of 85% to 120%, so the maximum model value of the 3D emotional coordinate system is (40, 150, 120), and the minimum model value thereof is (26, 75, 85). All the deduced situations generally fall within the two model values.

Each of the emotional phases 132 represents an emotional expression. As shown in FIG. 3, it is analyzed that positions (ψ1 and ψ2) of the bio-signals are both at an emotional phase 132 in the 3D emotional coordinate system, and the vector coordinates thereof reflect that the values of the finger temperature, heart rate, SpO2 are rising in positive directions (T+, P+, O+), so this emotional phase is defined as “excitement”. Therefore, when the positions (ψ1 and ψ2) of the bio-signals are both at the emotional phase 132 of “excitement”, the finger temperature, heart rate, and SpO2 of the individual may gradually rise due to external or internal factors, such that an emotional state of “excitement” is generated and the individual reacts in the same emotion. The emotion analysis module 120 acquires the emotional state information 131 of “excitement” from the emotion database 130 according to the determination result.

A media information 133 corresponding to the emotional state information 131 is outputted (Step S230). After obtaining the emotional state information 131, the emotion analysis module 120 acquires at least one medical advice 134 of the individual from the emotion database 130, and analyzes the emotional state information 131 with the medical advice 134, so as to obtain the media information 133 corresponding to the emotional state information 131.

The medical advice 134 stores emotional reactions and variations that the individual may show or should avoid, and establishes optimal models for the bio-signals from the individual in the 3D emotional coordinate system. The emotion analysis module 120 analyzes the emotional state information 131 through a stochastic approximation method, so as to obtain optimal parameters of the medical advice 134 matching the emotional state information 131, and to calculate and output the media information 133 suitable for the optimal parameters. After that, the media information 133 is played by the media player 140 to the individual (Step S240), for releasing the emotion of the individual.

Finally, the media information 133 is modified according to the variation range of the bio-signals (Step S250). After the media information 133 is outputted by the emotion analysis module 120 and played on the media player 140, the emotion analysis module 120 continues to analyze the bio-signals and to calculate the variation range of the bio-signals by the self-training module thereof, so as to obtain an emotional quotient.

In addition, the emotional quotient may be a positive or negative emotional quotient. First, the self-training module determines whether the emotional quotient is negative, and if not, it indicates that the currently played media information 133 is able to release the emotion of the individual. On the contrary, it indicates that the currently played media information 133 is unable to release the emotion of the individual. Accordingly, all the media information 133 corresponding to the current emotional state information 131 is gathered, and then set their priorities based on the matching degree from the optimal settings of the medical advice 134. After that, the media information 133 with the highest priority is obtained, and the media information 133 of the current emotional state information 131 is updated. Then, the update result is stored in the monitoring record 135 of the emotion database 130. In this manner, when the appropriate media information 133 is re-calculated by the emotion analysis module 120 or the self-training module and is re-played, the previously inappropriate media information 133 is eliminated. Finally, the bio-signals are continuously analyzed and the variation ranges thereof are calculated.

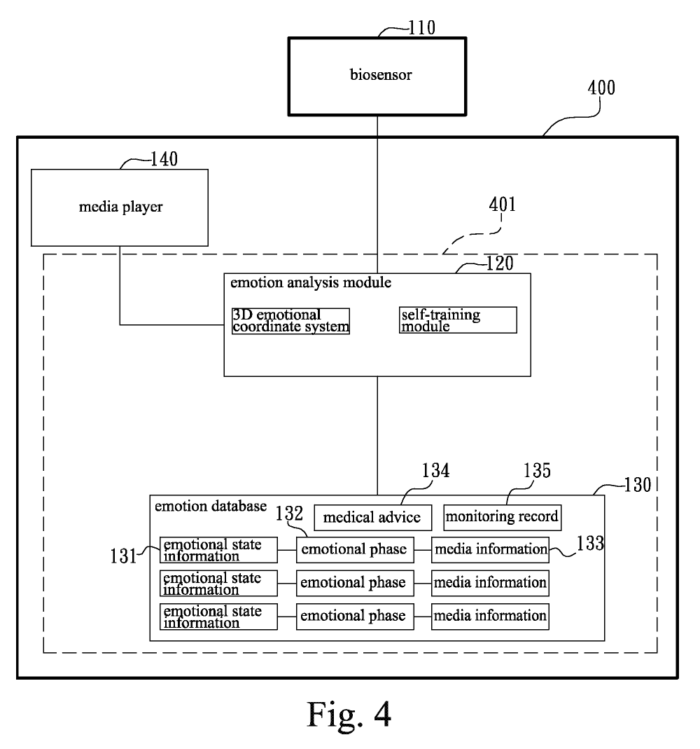

Referring to FIG. 4, a schematic view of a first configuration of the system according to an embodiment of the present invention is shown. The system is applied to a portable mobile apparatus 400, such as an MP3 player, personal digital assistant (PDA), hand-held phone, or hand-held computer. These mobile apparatus 400 are all characterized in having an embedded system, and the media player 140 is disposed on the mobile apparatus 400.

The biosensor 110 is carried by the individual, and has an appearance like a finger ring, wrist ring, neck ring, arm ring, or foot ring that can directly contact the skin of the individual. The biosensor mainly functions by sensing a plurality of types of bio-signals from the individual (such as finger temperature, heart rate, and SpO2) simultaneously with a broadband infrared sensor.

The media player 140 is disposed on the mobile apparatus 400. The emotion analysis module 120 and the emotion database 130 are configured in an embedded system 401, and are driven by the embedded system 401. The biosensor 110 is connected with the mobile apparatus 400 in a wired or wireless manner. In addition, the wired manner is fulfilled through a USB connection line or network connection. The wireless manner is fulfilled by mounting a wireless signal transmitter on the biosensor 110, and a wireless signal receiver on the mobile apparatus 400 to receive wireless signals transmitted by the biosensor 110. The applied wireless technique may be infrared transmitting/receiving technique, ZigBee communication network (such as 802.15.4), BlueTooth technique, or wireless local area network protocol (such as 802.11B).

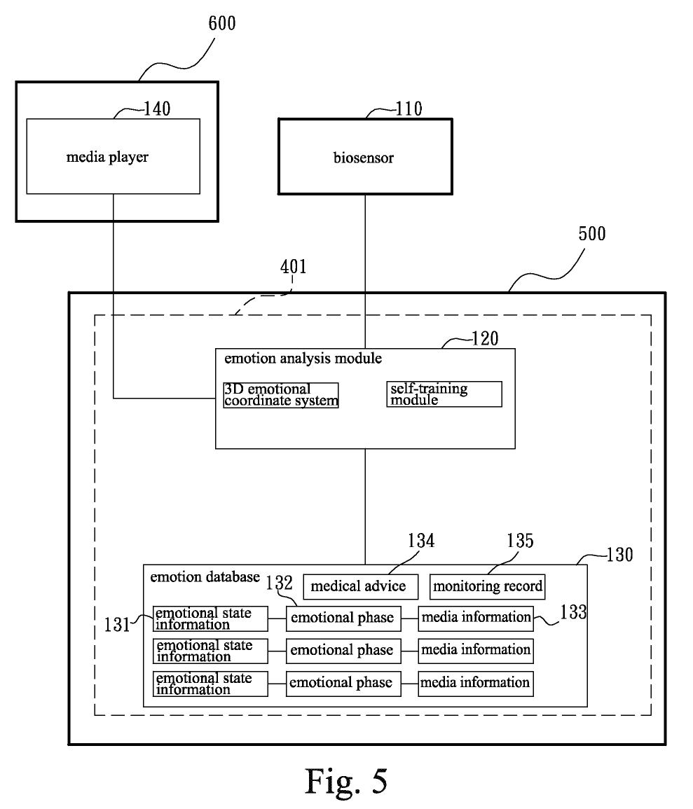

Referring to FIG. 5, a schematic view of a second configuration of the system according to an embodiment of the present invention is shown. Similarly, the system is also applied to a portable mobile apparatus 500. However, the media player 140 is not disposed on the mobile apparatus 500, but is an external apparatus 600, such as a desk-top PC, optical disc drive for CD/DVD, audio apparatus, or network apparatus for playing the media information 133.

As shown in FIG. 5, the biosensor 110 is carried by the individual. The emotion analysis module 120 and the emotion database 130 are configured in the embedded system 401. The biosensor 110 is connected with the mobile apparatus 500 in a wired or wireless manner, and the mobile apparatus 500 is also connected with the external apparatus 600 in a wired or wireless manner.

After analyzing the bio-signals to determine the corresponding emotional state information 131, the biosensor 110 acquires the relative media information 133 and stores the determination result in a storage space of the emotion database 130 or mobile apparatus. As such, when being connected with the external apparatus in a wired or wireless manner, the mobile apparatus 500 transmits the media information 133 to the external apparatus 600 in an active or passive manner according to the determination result, and thus the media information 133 is played by the media player 140.

Though the present invention has been disclosed above by the preferred embodiments, they are not intended to limit the present invention. Anybody skilled in the art can make some modifications and variations without departing from the spirit and scope of the present invention. Therefore, the protecting range of the present invention falls in the appended claims.

What is claimed is:

1. A system for perceiving and relaxing emotions, comprising:
a biosensor, for detecting a plurality of types of bio-signals from an individual;
an emotion database, for storing a plurality of emotional state information and a plurality of media information;
a media player; and
an emotion analysis module, connected with the biosensor, the emotion database, and the media player, for analyzing the emotional state information corresponding to the bio-signals, and outputting the media information corresponding to the emotional state information, so as to be played by the media player.

2. The system for perceiving and relaxing emotions as claimed in claim 1, wherein the emotion database further stores at least one medical advice, and after obtaining the emotional state information corresponding to the bio-signals, the emotion analysis module analyzes the media information corresponding to the emotional state information based on the medical advice, and outputs the media information to the media player.

3. The system for perceiving and relaxing emotions as claimed in claim 2, wherein the emotion analysis module analyzes the bio-signals through a stochastic approximation method, so as to find out the emotional state information corresponding to the medical advice, and output the media information corresponding to the emotional state information.

4. The system for perceiving and relaxing emotions as claimed in claim 1, wherein the bio-signals comprise finger temperature, heart rate, and SpO2 (Oxyhemoglobin saturation by pulse oximetry) of the individual.

5. The system for perceiving and relaxing emotions as claimed in claim 1, wherein the emotion analysis module constructs a three-dimensional (3D) emotional coordinate system with the bio-signals as coordinate axes, so as to divide the 3D emotional coordinate system into a plurality of emotional phases according to the coordinate axes, and the emotion database further stores the emotional phase corresponding to each emotional state information, such that the emotion analysis module analyzes the emotional state information corresponding to the bio-signals.

6. The system for perceiving and relaxing emotions as claimed in claim 1, wherein after outputting the media information of the emotional state information, the emotion analysis module continues to analyze the bio-signals and calculate variation range thereof, so as to conclude an emotional quotient, and modify the media information corresponding to the emotional state information based on the emotional quotient.

7. A method for perceiving and relaxing emotions, at least comprising:
detecting a plurality of bio-signals from an individual;
analyzing the bio-signals to obtain a corresponding emotional state information;
outputting a media information corresponding to the emotional state information; and
playing the media information to the individual.

8. The method for perceiving and relaxing emotions as claimed in claim 7, wherein the bio-signals comprise finger temperature, heart rate, and SpO2 (Oxyhemoglobin saturation by pulse oximetry) of the individual.

9. The method for perceiving and relaxing emotions as claimed in claim 7, wherein the step of analyzing the bio-signals to obtain a corresponding emotional state information further comprises:
constructing a three-dimensional (3D) emotional coordinate system with the bio-signals as coordinate axes;
dividing the 3D emotional coordinate system into a plurality of emotional phases according to the coordinate axes;
setting each of the emotional phases corresponding to one of the emotional state information; and
calculating the emotional phase of the bio-signals and a distance from the emotional phase to origin of the 3D emotional coordinate system, so as to determine the emotional state information corresponding to the bio-signals.

10. The method for perceiving and relaxing emotions as claimed in claim 7, wherein the step of outputting a media information corresponding to the emotional state information further comprises:
obtaining the emotional state information;
obtaining at least one medical advice of the individual;
analyzing the emotional state information with the medical advice, so as to obtain the media information corresponding to the emotional state information; and
outputting the media information.

11. The method for perceiving and relaxing emotions as claimed in claim 10, wherein the step of analyzing the emotional state information with the medical advice further comprises:
analyzing the emotional state information through a stochastic approximation method; and
obtaining optimal parameters of the medical advice matching the emotional state information.

12. The method for perceiving and relaxing emotions as claimed in claim 7, further comprising a step of modifying the media information according to variation range of the bio-signals.

13. The method for perceiving and relaxing emotions as claimed in claim 12, wherein the step of modifying the media information according to variation range of the bio-signals further comprises:
analyzing the bio-signals and calculating the variation range thereof, so as to conclude an emotional quotient;
determining whether the emotional quotient is a negative emotional quotient, so as to obtain the media information with a highest priority from the media information corresponding to the emotional state information; and
updating the media information of the emotional state information and returning to the step of analyzing the bio-signals and calculating the variation range thereof.

14. A recording medium for perceiving and relaxing emotions, used for recording computer readable program codes to be executed on a system for perceiving and relaxing emotions of an individual by detecting emotional variations and releasing the emotions of the individual, comprising:
detecting a plurality of bio-signals from an individual;
analyzing the bio-signals to obtain a corresponding emotional state information;
outputting a media information corresponding to the emotional state information; and
playing the media information to the individual.

15. The recording medium as claimed in claim 14, wherein the bio-signals comprise finger temperature, heart rate, and SpO2 (Oxyhemoglobin saturation by pulse oximetry) of the individual.

16. The recording medium as claimed in claim 14, wherein the step of analyzing the bio-signals to obtain a corresponding emotional state information further comprises:
constructing a three-dimensional (3D) emotional coordinate system with the bio-signals as coordinate axes;
dividing the 3D emotional coordinate system into a plurality of emotional phases according to the coordinate axes;
setting each of the emotional phases corresponding to one of the emotional state information; and
calculating the emotional phase of the bio-signals and a distance from the emotional phase to origin of the 3D emotional coordinate system, so as to determine the emotional state information corresponding to the bio-signals.

17. The recording medium as claimed in claim 14, wherein the step of outputting a media information corresponding to the emotional state information further comprises:
obtaining the emotional state information;
obtaining at least one medical advice of the individual;
analyzing the emotional state information with the medical advice, so as to obtain the media information corresponding to the emotional state information; and
outputting the media information.

18. The recording medium as claimed in claim 17, wherein the step of analyzing the emotional state information with the medical advice further comprises:
analyzing the bio-signals through a stochastic approximation method; and
obtaining the emotional state information corresponding to the medical advice.

19. The recording medium as claimed in claim 14, further comprising a step of modifying the media information according to variation range of the bio-signals.

20. The recording medium as claimed in claim 19, wherein the step of modifying the media information according to the variation range of the bio-signals further comprises:
analyzing the bio-signals and calculating the variation range thereof, so as to conclude an emotional quotient;
determining whether the emotional quotient is a negative emotional quotient, so as to obtain the media information with a highest priority from the media information corresponding to the emotional state information; and
updating the media information of the emotional state information and returning to the step of analyzing the bio-signals and calculating the variation range thereof.
